(19) United States
(12) Patent Application Publication
(10) Pub. No.: US 2025/0285708 A1
(43) Pub. Date: Sep. 11, 2025

(54) MONITORING MOLECULAR RESPONSE BY ALLELIC IMBALANCE

(71) Applicant: GUARDANT HEALTH, INC., Palo Alto, CA (US)

(72) Inventors: Che-yu LEE, Redwood City, CA (US); Katie Julia QUINN, Redwood City, CA (US); Tingting JIANG, San Diego, CA (US)

(21) Appl. No.: 18/924,678

(22) Filed: Oct. 23, 2024

Related U.S. Application Data
(60) Provisional application No. 63/593,734, filed on Oct. 27, 2023.

(51) Int. Cl. G16B 20/20; G16B 30/10; G16H 20/10; G16H 50/20

(52) U.S. Cl. CPC G16B 20/20; G16B 30/10; G16H 20/10; G16H 50/20

(57) ABSTRACT

The present disclosure provides methods and systems for detecting an allelic imbalance molecular response in a sample from a subject. By determining allelic imbalance in single nucleotide polymorphisms (SNPs), a mutant allele fraction (MAF) measurement and divergence for SNPs in surrounding cluster and at gene level for all SNPs supporting determination of an allelic imbalance molecular response. Such determination increases evaluable samples that may otherwise be uninformative.

### CROSS-REFERENCE TO RELATED APPLICATION

The present application claims the benefit of U.S. Provisional Application No. 63/593,734, filed Oct. 27, 2023, which is fully incorporated by reference herein for all purposes.

### BACKGROUND

In cancer subjects (e.g., patients), allelic imbalance can be caused by loss of heterozygosity and can introduce a different distribution of mutant allele fraction (MAF) into assays of cell-free nucleic acid samples from a subject, as compared to samples without allelic imbalance. For example, a sample with allelic imbalance may have germline variants in very low MAF. Germline variants may also be observed with low MAF in cases where a sample is contaminated, such as during processing for sequencing, or where a sample has a second genome (other than the subject's genome) arising from, for example, a transplant, a blood transfusion, or a fetus.

Described herein is the use of allelic imbalance (AI) of common SNPs to calculate molecular response (MR). Examples of MR score, include that found in PCT App. No. PCT/US2023/079340, which is fully incorporated by reference herein. Some techniques involved comparing driver mutation MAF ratio between one or more time points. However, if no driver mutations is detected in a sample, sample is not evaluable for an MR score, and it is understood that MR score is affected by CHIP variants. When too many CHIP variants are detected, sample is not evaluable. Described herein are methods and compositions related to AI in determining MR score, which s not determined by single mutation, not affected by CHIP, reduce the rate of non- evaluable samples, with no CNV training required.

### SUMMARY

Recognized herein are challenges that may be encountered in distinguishing allelic imbalance samples from contaminated samples or samples containing a second genome. In cases where cell-free nucleic acids from samples containing contamination or a second genome are assayed, the samples may need additional manual review or even additional sequencing runs to be performed. As a result, failure to distinguish allelic imbalance samples from contaminated or second genome samples may significantly increase the cost and turn-around time of reliably assaying such samples. The present disclosure provides methods and systems to identify allelic imbalance or contamination in cell-free nucleic acid samples. Such methods and systems may obtain and analyze quantitative measures of small variant and copy number variation to identify the allelic imbalance or contamination. The use allelic imbalance (AI) of common SNPs to calculate MR provides opportunities for determining MR score that is not determined by single mutation, unaffected by CHIP, reduce the rate of sample being not evaluable, and without CNV training required.

Described herein is a method including receiving a plurality of sequence reads from a subject, aligning the sequence reads to a reference, determining one or more metrics based on the alignment to the reference, and processing the one or more metrics generated from the aligned reads to determine a sample score. In other embodiments, the method includes obtaining a sample from the subject, and sequencing the sample to obtain the plurality of sequence reads. In other embodiments, the method includes one or more metrics comprise a plurality of single nucleotide polymorphisms (SNPs). In other embodiments, the method includes SNPs comprise common SNPs. In other embodiments, the method includes SNPs have a minor allele frequency (MAF) ranging from 5% to 95%. In other embodiments, the method includes, from the plurality of SNPs, at least one allelic imbalance cluster. In other embodiments, the method includes SNPs with MAF less than 45% or greater than 55%. In other embodiments, the method includes at least one allelic imbalance cluster comprise SNPs with a MAF either less than 50% or greater than 50%. In other embodiments, the method includes at least one allelic imbalance clusters comprise at least one SNPs. In other embodiments, the method includes at least one allelic imbalance clusters comprise five or more SNPs. In other embodiments, the method includes sample score includes determining a MAF shift score for each SNP. In other embodiments, the method includes MAF shift score includes the absolute difference between MAF for a given SNP and 50%. In other embodiments, the method includes, a gene-level MAF shift score. In other embodiments, the method includes gene-level MAF shift score includes the median, second quartile, mean, weighted mean, geometric mean, harmonic mean, or winsorized mean MAF shift score of all the SNPs in the same gene. In other embodiments, the method includes sequence reads include data from a plurality of samples collected at different timepoints from the same subject. In other embodiments, the method includes an allelic imbalance molecular response score (Ai MR score), such as when AI MR=median(gene-level maf shift score at T1)/median(gene-level maf shift score at T0) and/or optionally based on Formula 1 . . . In other embodiments, the method is an ensemble method including for example, a composite final score, based on one or more of the methods described herein (MR, AI MR, AI MR-CNV).

In other embodiments, the method includes Ai MR score includes the ratio of the median gene-level MAF shift score at T2 to the median gene-level MAF shift score at T1. In other embodiments, the method includes Ai MR score includes the ratio of the median gene-level MAF shift score at Ti to the median gene-level MAF shift score at Tj for a given pair of timepoints (Ti and Tj). In other embodiments, the method includes timepoint Ti includes a baseline and Tj includes a subsequent timepoint following the baseline. In other embodiments, the method includes Tj includes timepoints T1, T2, T3 . . . Tn, where “n” represents the set of all real numbers greater than or equal to 1, i.e., [1, ∞). In other embodiments, the method includes Tj is compared to timepoint Ti. In other embodiments, the method includes baseline timepoint Ti is a timepoint before treating the subject with a therapy drug and the subsequent timepoint Tj is a timepoint after the treating the subject with the therapy. In other embodiments, the method includes sample includes cell free nucleic acids (cf NA). In other embodiments, the method includes cf NA are cell free DNA or cell free RNA.

In other embodiments, the method includes wherein the subject is a cancer patient. In other embodiments, the method includes cancer is selected from the group consisting of biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemiaF (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, and uterine sarcoma.

Described herein is a computer implemented method including receiving a plurality of sequence reads including data from a plurality of samples from the same subject, aligning the sequence reads to a reference, processing the sequence reads to determine a plurality of allelic imbalance clusters including at least one SNP, determining a MAF shift score for each SNP in the cluster, determining a gene-level MAF shift scores for all the SNPs in the same gene, and processing the gene-level MAF shift scores to determine an allelic imbalance molecular response score (Ai MR score) for the plurality of samples from the patient, AI MR=median (gene-level maf shift score at T1)/median(gene-level maf shift score at T0), optionally based on Formula 1. In other embodiments, the method is an ensemble method including for example, a composite final score, based on one or more of the methods described herein (MR, AI MR, AI MR-CNV). t. In other embodiments, the method includes obtaining a plurality of samples from the subject, wherein at least one sample from the plurality of samples is obtained before administering a therapy to the subject, and sequencing the plurality of samples to obtain the plurality of sequence reads.

In other embodiments, the method includes therapy is selected from the group consisting of an immune checkpoint inhibitor, poly (ADP-ribose) polymerase (PARP) inhibitor, a kinase inhibitor, or an aromatase inhibitor, a PI3K and mTOR inhibitor. In other embodiments, the method includes immune checkpoint inhibitor includes Pembrolizumab. In other embodiments, the method includes the poly (ADP-ribose) polymerase (PARP) inhibitor Olaparib or Talazoparib. In other embodiments, the method includes therapy is a combination of a PI3K and mTOR inhibitor and a poly (ADP-ribose) polymerase (PARP) inhibitor. In other embodiments, the method includes PI3K and mTOR inhibitor includes Gedatolisib and the poly (ADP-ribose) polymerase (PARP) inhibitor includes Talazoparib. In other embodiments, the method includes determining a therapeutic response in the subject when the Ai MR score falls below a predefined threshold. In other embodiments, the method includes Ai MR score of 1 indicates unchanged tumor fraction, Ai MR score greater than 1 indicates increased tumor fraction, and Ai MR less than 1 indicates decrease tumor fraction. In other embodiments, the method includes an Ai MR less than 0.5 indicates a therapeutic response. In other embodiments, the method includes an Ai MR less than 1 indicates a therapeutic response. In other embodiments, the method includes a sample is selected from the group consisting of blood, serum, plasma, bone marrow aspirate, bile, cerebral spinal fluid (CSF), saliva, and urine.

In some embodiments, determination of allelic imbalance in a cluster, genomic locus, or otherwise includes: (a) sequencing a plurality of cell-free nucleic acid molecules from the sample to generate a plurality of sequence reads, (b) aligning at least a portion of the plurality of sequence reads to a reference sequence to produce a plurality of aligned sequence reads, (c) for at least a portion of the plurality of aligned sequence reads, identifying a germline variant present at a mutant allele fraction (MAF) in the sample, thereby identifying a set of germline variants in the sample, wherein individual germline variants in the set of germline variants have corresponding MAF values, (d) determining a quantitative measure of the set of germline variants identified in (c) that are among a plurality of discrete ranges of MAF values, and (e) detecting the presence or absence of the allelic imbalance in the sample based on a predetermined criterion by filtering the set of germline variants identified in (c) based on at least the quantitative measure of (d).

In some embodiments, determination of allelic imbalance (a) sequencing a plurality of cell-free deoxyribonucleic acid (DNA) molecules from the sample to generate a plurality of sequence reads; (b) aligning at least a portion of the plurality of sequence reads to a reference sequence to produce a plurality of aligned sequence reads; (c) for at least a portion of the plurality of aligned sequence reads, identifying a germline variant present at a mutant allele fraction (MAF) in the sample, thereby identifying a set of germline variants in the sample, wherein individual germline variants in the set of germline variants have corresponding MAF values; (d) determining a quantitative measure of the set of germline variants identified in (c) that are among a plurality of discrete ranges of MAF values; and (e) detecting the presence or absence of the allelic imbalance in the sample based on a predetermined criterion by filtering the set of germline variants identified in (c) based on at least the quantitative measure of (d).

In some embodiments, the detecting in (e) includes detecting, from the plurality of aligned sequence reads, one or more quantitative measures indicative of copy number variations (CNVs) or diploid genes, wherein the predetermined criterion includes the one or more quantitative measures indicative of the CNVs or the diploid genes.

In some embodiments, the method further includes detecting a presence or absence of contamination or a second genome in the sample when the absence of the allelic imbalance is detected in the sample.

In some embodiments, the set of germline variants includes at least about 50, at least about 100, at least about 200, at least about 500, at least about 1,000, at least about 2,000, at least about 5,000, at least about 10,000, or more than about 10,000 distinct germline variants. In some embodiments, the set of genetic variants includes genetic variants selected from the group consisting of a single nucleotide variant (SNV), an insertion or deletion (indel), and a fusion. In some embodiments, the sample is a bodily fluid sample selected from the group consisting of blood, plasma, serum, urine, saliva, mucosal excretions, sputum, stool, and tears. In some embodiments, the subject has a disease or disorder. In some embodiments, the disease is cancer.

In some embodiments, the method further includes amplifying the cell-free DNA molecules prior to sequencing. In some embodiments, the method further includes selectively enriching the cell-free DNA molecules for a set of genetic loci prior to sequencing. In some embodiments, the method further includes attaching one or more adapters including barcodes to the cell-free DNA molecules prior to sequencing. In some embodiments, the one or more adapters are randomly attached to both ends of the cell-free DNA molecules. In some embodiments, the cell-free DNA molecules are uniquely barcoded. In some embodiments, the cell-free DNA molecules are non-uniquely barcoded. In some embodiments, each barcode includes a fixed or semi-random oligonucleotide sequence that in combination with a diversity of molecules sequenced from a selected region enables identification of unique cell-free DNA molecules. In some embodiments, the plurality of genomic regions includes genetic variants found in COSMIC, The Cancer Genome Atlas (TCGA), or the Exome Aggregation Consortium (ExAC). In some cases, genetic variants may belong to a pre-defined set of clinically actionable variants. For example, such variants may be found in various databases of variants whose presence in a sample of a subject have been shown to correlate with or be indicative of a disease or disorder (e.g., cancer) in the subject. Such databases of variants may include, for example, the Catalogue of Somatic Mutations in Cancer (COSMIC), The Cancer Genome Atlas (TCGA), and the Exome Aggregation Consortium (ExAC). In some embodiments, the plurality of genomic regions includes a BRCA1 genetic variant (e.g., BRCA1 P209L). A pre-defined set of such catalogued variants may be designated for further bioinformatics analysis due to their relevance to clinical decision-making (e.g., diagnosis, prognosis, treatment selection, targeted treatment, treatment monitoring, monitoring for recurrence, etc.). Such a pre-defined set may be determined based on, for example, analysis of clinical samples (e.g., of patient cohorts with known presence or absence of a disease or disorder) as well as annotation information from public databases and clinical literature.

In some embodiments, the plurality of discrete ranges of MAF values includes a first range of about 3% to about 40% and a second range of about 60% to about 97%. In some embodiments, the quantitative measure of (d) includes a number of the set of genetic variants that are among the plurality of discrete ranges of MAF values. In some embodiments, the predetermined criterion includes the quantitative measure of (d) being greater than a predetermined germline variant threshold. In some embodiments, the predetermined germline variant threshold is about 21. In some embodiments, the one or more quantitative measures indicative of the CNVs or the diploid genes are selected from the group consisting of a maximum CNV level across the sample, a minimum CNV level across the sample, a fraction of diploid genes, and a copy number mean. In some embodiments, the one or more quantitative measures indicative of the CNVs or the diploid genes comprise two or more quantitative measures selected from the group consisting of a maximum CNV level across the sample, a minimum CNV level across the sample, a fraction of diploid genes, and a copy number mean. In some embodiments, the one or more quantitative measures indicative of the CNVs or the diploid genes comprise three or more quantitative measures selected from the group consisting of a maximum CNV level across the sample, a minimum CNV level across the sample, a fraction of diploid genes, and a copy number mean. In some embodiments, the predetermined criterion includes one or more criteria selected from the group consisting of: a maximum CNV level across the sample of greater than a predetermined maximum CNV threshold, a minimum CNV level across the sample of less than a predetermined minimum CNV threshold, a fraction of diploid genes of less than a predetermined fraction diploid threshold, and a copy number mean in the same germline variant having an absolute value greater than a predetermined copy number mean threshold, wherein the same germline variant has an MAF of less than about 3%. In some embodiments, the predetermined criterion includes two or more criteria selected from the group consisting of: a maximum CNV level across the sample of greater than a predetermined maximum CNV threshold, a minimum CNV level across the sample of less than a predetermined minimum CNV threshold, a fraction of diploid genes of less than a predetermined fraction diploid threshold, and a copy number mean in the same germline variant having an absolute value greater than a predetermined copy number mean threshold, wherein the same germline variant has an MAF of less than about 3%. In some embodiments, the predetermined criterion includes three or more criteria selected from the group consisting of: a maximum CNV level across the sample of greater than a predetermined maximum CNV threshold, a minimum CNV level across the sample of less than a predetermined minimum CNV threshold, a fraction of diploid genes of less than a predetermined fraction diploid threshold, and a copy number mean in the same germline variant having an absolute value greater than a predetermined copy number mean threshold, wherein the same germline variant has an MAF of less than about 3%. In some embodiments, the predetermined criterion includes a maximum CNV level across the sample of greater than a predetermined maximum CNV threshold, a minimum CNV level across the sample of less than a predetermined minimum CNV threshold, a fraction of diploid genes of less than a predetermined fraction diploid threshold, and a copy number mean in the same germline variant having an absolute value greater than a predetermined copy number mean threshold, wherein the same germline variant has an MAF of less than about 3%. In some embodiments, the predetermined criterion includes one or more thresholds selected from the group consisting of: a maximum CNV threshold of about 0.22, a minimum CNV threshold of about −0.14, a fraction diploid threshold of about 0.7, and a copy number mean threshold of about 10. In some embodiments, the predetermined criterion includes two or more thresholds selected from the group consisting of: a maximum CNV threshold of about 0.20, about 0.21, or 0.22; a minimum CNV threshold of about −0.10, about −0.11, about −0.12, about −0.13, about −0.14, or about −0.15; a fraction diploid threshold of about 0.5, about 0.6, about 0.7, about 0.8, about 0.9, about 0.10; and a copy number mean threshold of about 5, about 6, about 7, about 8, about 9, about 10, or about 15. In some embodiments, the predetermined criterion includes three or more thresholds selected from the group consisting of: a maximum CNV threshold of about 0.22, a minimum CNV threshold of about −0.14, a fraction diploid threshold of about 0.7, and a copy number mean threshold of about 10. In some embodiments, the predetermined criterion includes a maximum CNV threshold of about 0.22, a minimum CNV threshold of about −0.14, a fraction diploid threshold of about 0.7, and a copy number mean threshold of about 10.

In some embodiments, the method further includes detecting the presence of the contamination or the second genome in the sample with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In some embodiments, the method further includes detecting the absence of the contamination or the second genome in the sample with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In some embodiments, the PPV and/or NPV are determined based on testing data from a training set of samples (e.g., about 10 samples, about 20 samples, about 30 samples, about 40 samples, about 50 samples, about 100 sample, about 150 samples, about 200 samples, or about 250 samples) whose contamination/allele imbalance status is known.

In some embodiments, the method further includes detecting the presence of the contamination or the second genome in the sample with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the method further includes detecting the absence of the contamination or the second genome in the sample with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the method further includes identifying the germline variant by: (i) determining a total allele count and a mutant allele count for a nucleic acid variant from the cfDNA molecules; (ii) identifying an associated variable of the nucleic acid variant from the cfDNA molecules; (iii) determining a quantitative value for the associated variable of the nucleic acid variant; (iv) generating a statistical model for expected germline mutant allele counts at a genomic locus of the nucleic acid variant; (v) generating a probability value (p-value) for the nucleic acid variant based at least in part on the statistical model for expected germline mutant allele counts, the quantitative value for the associated variable of the nucleic acid variant, and at least one of the total allele count and the mutant allele count for the nucleic acid variant; and (vi) classifying the nucleic acid variant as (1) being of somatic origin when the p-value for the nucleic acid variant is below a predetermined threshold value, or as (2) being of germline origin when the p-value for the nucleic acid variant is at or above the predetermined threshold value.

In some embodiments, the method further includes detecting in a cluster, genomic locus, or otherwise based on at least one of the set of germline variants identified in (c) as present at a given MAF. In some embodiments, the allele-specific loss in the sample is detected based on the at least one of the set of germline variants being present at an MAF below 50% in the sample from the subject. In some embodiments, the allele-specific loss in the sample is detected based on the at least one of the set of germline variants being present at an MAF below 50% in the sample from the subject and in each of one or more samples from one or more additional subjects. In some embodiments, the at least one of the set of germline variants is found in COSMIC, The Cancer Genome Atlas (TCGA), or the Exome Aggregation Consortium (ExAC). In some embodiments, the at least one of the set of germline variants is a BRCA1 gene variant. In some embodiments, the BRCA1 gene variant is BRCA1 P209L.

In another aspect, the present disclosure provides a system, including a controller including, or capable of accessing, computer readable media including non-transitory computer-executable instructions which, when executed by at least one electronic processor, perform at least: (a) obtaining a plurality of sequence reads corresponding to a plurality of cell-free deoxyribonucleic acid (DNA) molecules from a sample of a subject; (b) aligning at least a portion of the plurality of sequence reads to a reference sequence to produce a plurality of aligned sequence reads; (c) for at least a portion of the plurality of aligned sequence reads, identifying a germline variant present at a mutant allele fraction (MAF) in the sample, thereby identifying a set of germline variants in the sample, wherein individual germline variants in the set of germline variants have corresponding MAF values; (d) determining a quantitative measure of the set of germline variants identified in (c) that are among a plurality of discrete ranges of MAF values; and (e) detecting the presence or absence of allelic imbalance in the sample based on a predetermined criterion by filtering the set of germline variants identified in (c) based on at least the quantitative measure of (d).

In some embodiments, the detecting in (e) further includes detecting, from the plurality of aligned sequence reads, one or more quantitative measures indicative of copy number variations (CNVs) or diploid genes, wherein the predetermined criterion includes the one or more quantitative measures indicative of the CNVs or the diploid genes. In some embodiments, the system further includes a nucleic acid sequencer operably connected to the controller, which nucleic acid sequencer is configured to process the plurality of cell-free DNA molecules from the sample to generate the plurality of sequence reads.

In some embodiments, the non-transitory computer-executable instructions, when executed by at least one electronic processor, further perform generating a report which optionally includes information on the presence or absence of the allelic imbalance of the sample and/or information on the presence or absence of the contamination or second genome of the sample. In some embodiments, the non-transitory computer-executable instructions, when executed by at least one electronic processor, further perform communicating the report to a third party, such as the subject from whom the sample is derived or a health care practitioner.

In an aspect, the present disclosure provides a method for detecting a presence or absence of an allelic imbalance in a sample from a subject, including: (a) accessing, by a computer system, a plurality of sequencing reads generated from a plurality of cell-free deoxyribonucleic acid (DNA) molecules from the sample to; (b) aligning, by the computer system, at least a portion of the plurality of sequence reads to a reference sequence to produce a plurality of aligned sequence reads; (c) for at least a portion of the plurality of aligned sequence reads, identifying, by the computer system, a germline variant present at a mutant allele fraction (MAF) in the sample, thereby identifying a set of germline variants in the sample, wherein individual germline variants in the set of germline variants have corresponding MAF values; (d) determining, by the computer system, a quantitative measure of the set of germline variants identified in (c) that are among a plurality of discrete ranges of MAF values; and (e) detecting, by the computer system, the presence or absence of the allelic imbalance in the sample based on a predetermined criterion by filtering the set of germline variants identified in (c) based on at least the quantitative measure of (d).

In some embodiments, the detecting in (e) includes (f) detecting, by the computer system, one or more quantitative measures indicative of copy number variations (CNVs) or diploid genes from the plurality of aligned sequence reads, wherein the predetermined criterion includes the one or more quantitative measures indicative of the CNVs or the diploid genes.

In some embodiments, the method further includes generating a report which optionally includes information on the presence or absence of the allelic imbalance of the sample and/or information on the presence or absence of the contamination or second genome of the sample. In some embodiments, the method further includes communicating the report to a third party, such as the subject from whom the sample is derived or a health care practitioner.

Another aspect of the present disclosure provides a non-transitory computer readable medium including machine executable code that, upon execution by one or more computer processors, implements any of the methods above or elsewhere herein.

Another aspect of the present disclosure provides a system including one or more computer processors and computer memory coupled thereto. The computer memory includes machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF FIGURES

FIG. 1. Sample workflow for calculating molecular response.

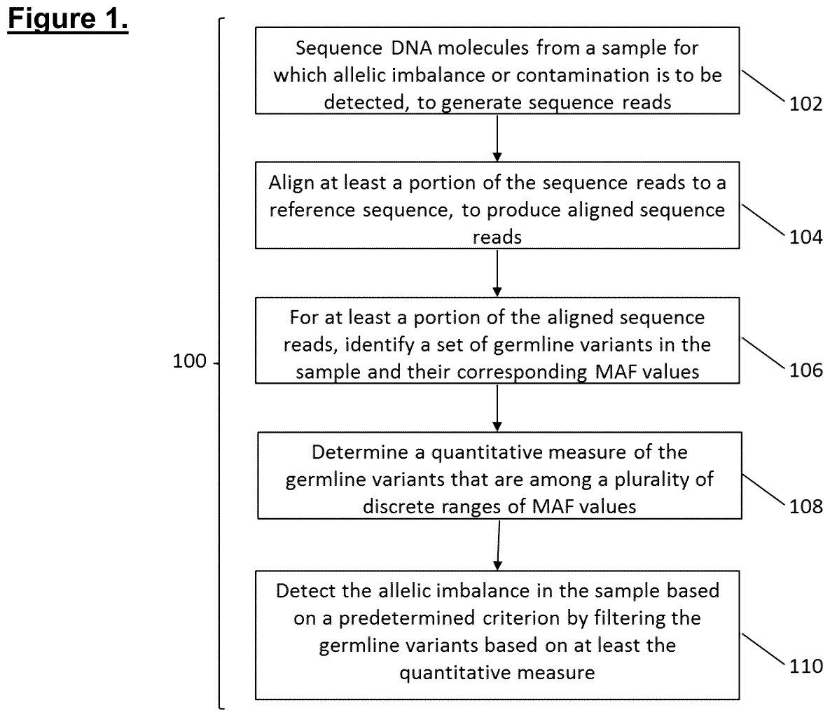

FIG. 2. Allelic Imbalance can predict tumor fraction

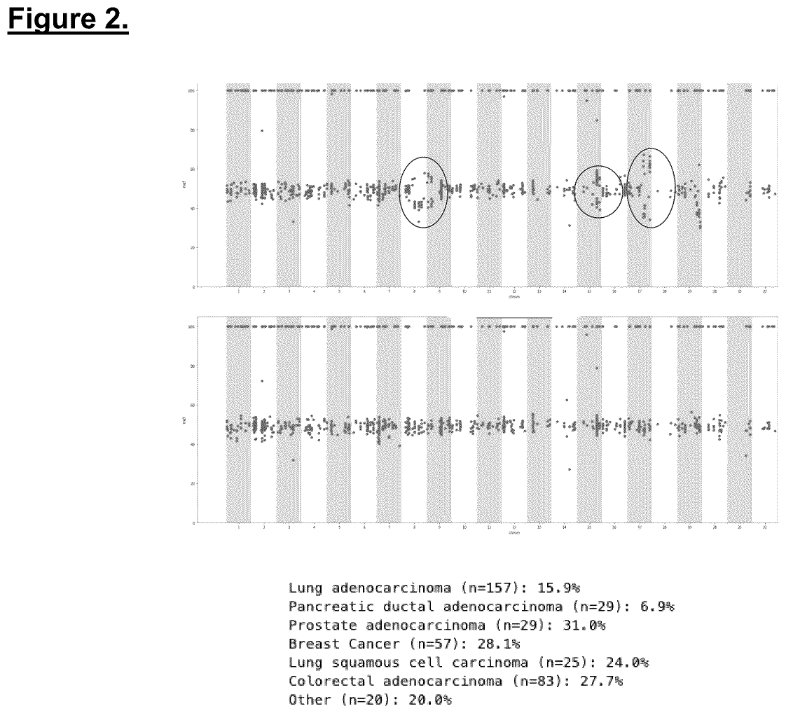

FIG. 3. Allelic Imbalance vs CNV call

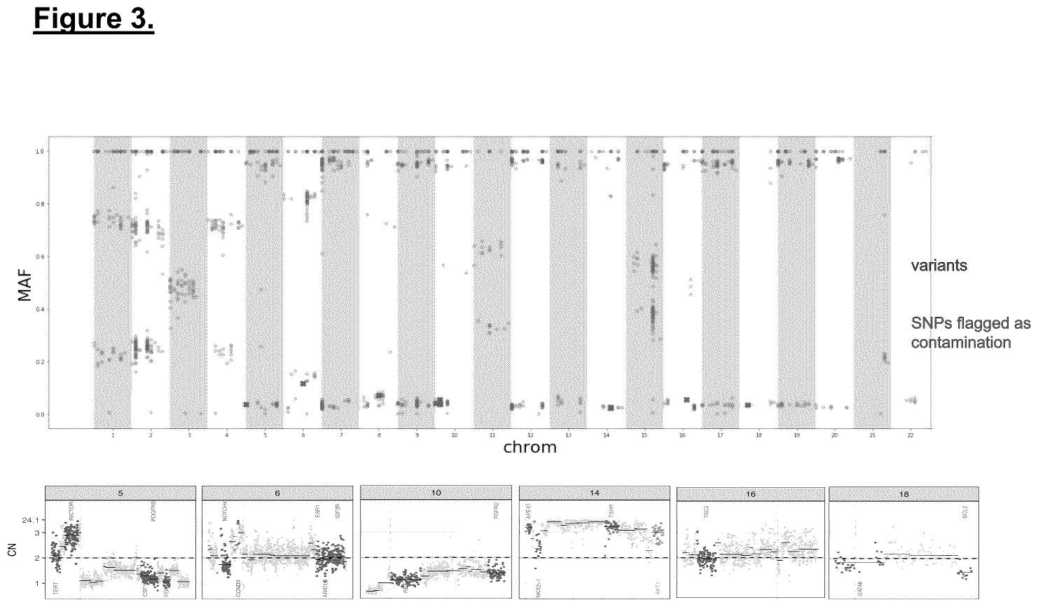

FIG. 4. AI MR Score and MR Score. Comparison of AI-based MR score and MR score, which are similar in most samples. As 2 score systems can have the same scale, comparisons avoid errors.

FIG. 5. Increase the Evaluable samples. In a cohort of 373 samples, 135 samples were not evaluable under existing MR score. There were 104 samples with allelic imbalance with at least 15 not evaluable samples can be evaluabled by AI MR. Some MR not-evaluable samples have high AI MR score. FIG. 5A depicts allele imbalance samples with current MR score. FIG. 5B depicts AI MR score of MR not-evaluable samples.

FIG. 6. Increase the Evaluable samples. AI MR score predicts better survival rate. FIG. 6A depicts allele imbalance samples with current MR score. FIG. 6B depicts AI MR score. FIG. 6C depicts survival rate on conflicting sampes.

FIG. 7. New MR score design workflow.

FIG. 8. epi/somatic MR score resolution. Depicted are methods to resolve the conflict prediction between epi/somatic MR, observations are at day 1, top panel, and day 15, bottom panel.

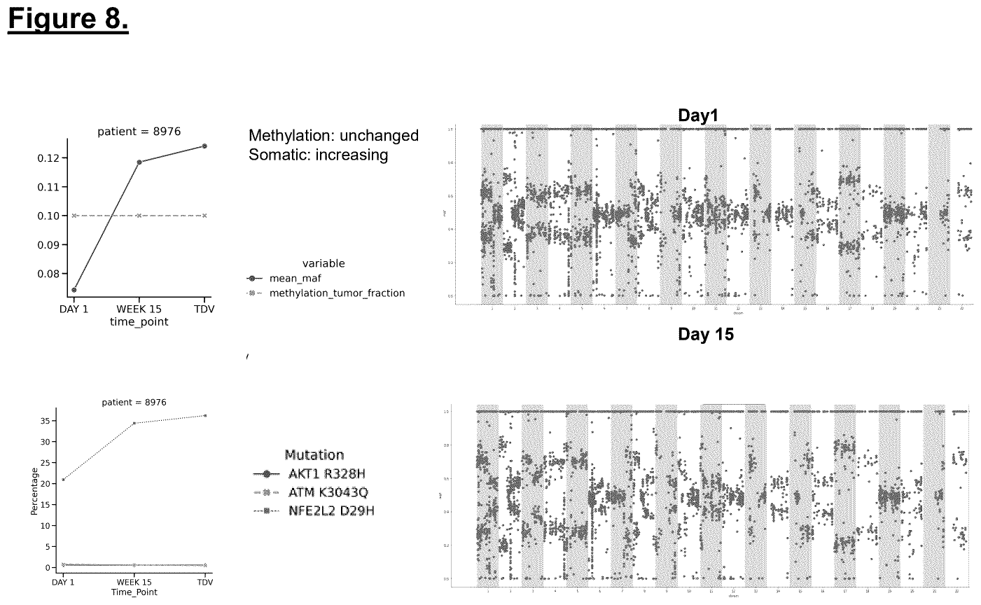

FIG. 9. An additional MR based on CNV formula, Formula 1.

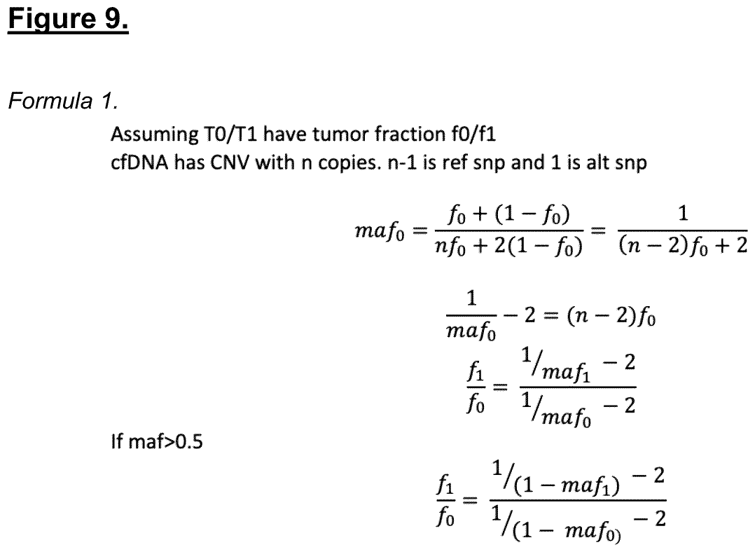

FIG. 10. AI MR Score vs MR Score.

FIG. 11. AI MR score in CNV formula. In some instances, when the AI is high, the CNV based tumor fraction (TF) is higher than somatic based TF

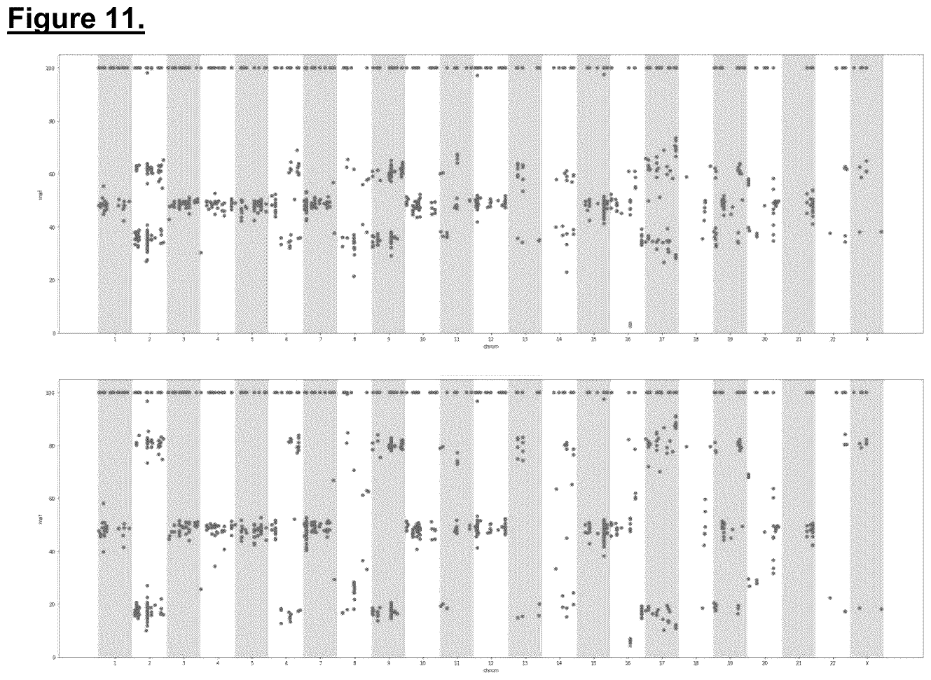

FIG. 12. NSCLC data. Cohort drawn from non-small cell lung cancer (NSCLC) wherein only 1 out of 177 samples not evaluable under conventional MR scoring. Comparison of scoring systems.

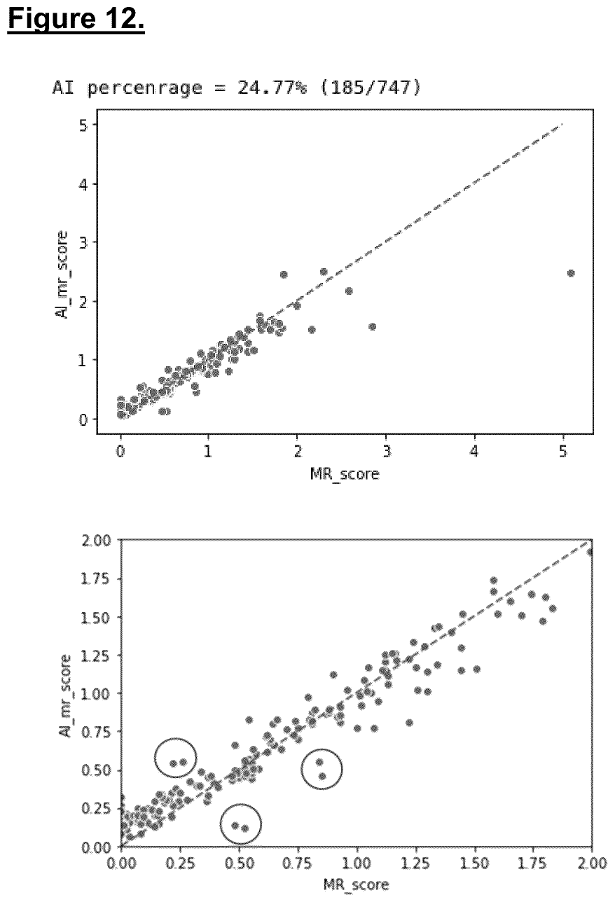

FIG. 13. NSCLC data. Cohort drawn from non-small cell lung cancer (NSCLC) showing comparison between AI MR, left panel, and CNV based AI MR, right panel.

### DETAILED DESCRIPTION

### I. Overview

In cancer patients, allelic imbalance can be caused by loss of heterozygosity and can introduce a different distribution of mutant allele fraction (MAF) into assays of cell-free nucleic acid samples from a subject, as compared to samples without allelic imbalance. For example, a sample with allelic imbalance may have germline variants in very low MAF. Germline variants may also be observed with low MAF in cases where a sample is contaminated, such as during processing for sequencing, or where a sample has a second genome (other than the subject's genome) arising from, for example, a transplant, a blood transfusion, or a fetus. Therefore, challenges may be encountered in distinguishing allelic imbalance samples from contaminated samples or samples containing a second genome.

In cases where cell-free nucleic acids from samples containing contamination or a second genome are assayed, the samples may need additional manual review or even additional sequencing runs to be performed. As a result, failure to distinguish allelic imbalance samples from contaminated or second genome samples may significantly increase the cost and turn-around time of reliably assaying such samples. The present disclosure provides methods and systems to identify allelic imbalance or contamination in cell-free nucleic acid samples. Such methods and systems may obtain and analyze quantitative measures of small variant and copy number variation to identify the allelic imbalance or contamination.

In various embodiments, allelic imbalance (also “allele imbalance”) generally refers to a difference in the DNA levels between two alleles in a gene (e.g., as a result of Loss of Heterozygosity). Allelic imbalance may occur in cases where a ratio of DNA levels between two alleles in a gene is not about 1. For example, allelic imbalance may arise as a result of gene imprinting, where epigenetics and environmental factors may affect the expression of one or both alleles in a given gene. As another example, cis-acting mutations may affect regulation of one allele among a pair of alleles in a gene, such as through changes in promoter or enhancer regions (e.g., transcription factor binding sites) or to 3′ UTR regions.

Loss of Heterozygosity (LOH) is a form of allelic imbalance in which one allele of an allele pair at a genetic locus is completely lost. LOH can arise via a number of genetic mechanisms, such as physical deletion, chromosome nondisjunction, mitotic nondisjunction followed by reduplication of the remaining chromosome, mitotic recombination, and gene conversion. LOH can be detected based on measurements of mutant allele fraction or minor allele frequency at a genetic locus. LOH may arise, for example, in cases where a tumor suppressor gene is inactivated such that one allele of the tumor suppressor gene allele pair is mutated and the other allele is lost.

In various embodiments, minor allele frequency is the frequency at which minor alleles (e.g., not the most common allele) occurs in a given population of nucleic acids, such as a sample obtained from a subject. Genetic variants at a low minor allele frequency typically have a relatively low frequency of presence in a sample.

In various embodiments, mutant allele count is the number of nucleic acid molecules among a plurality of nucleic acid molecules (e.g., obtained or derived from a sample) which are harboring a mutant allele or allelic alteration at a particular genomic locus.

In various embodiments, a mutant allele fraction (e.g., “mutation dose,” or “MAF”) refers to the fraction of nucleic acid molecules harboring an allelic alteration or mutation at a given genomic position in a given sample. MAF is generally expressed as a fraction or a percentage. For example, an MAF is typically less than about 0.5, 0.1, 0.05, or 0.01 (i.e., less than about 50%, 10%, 5%, or 1%) of all somatic variants or alleles present at a given locus.

In various embodiments, a nucleic acid tag includes a short nucleic acid (e.g., less than n nucleotides in length, where n is about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag includes a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples including nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, less than about a 0.1%, less than about a 0.01%, less than about a 0.001%, or less than about a 0.0001% chance) that any two molecules may have the same endogenous sequence information (e.g., start and/or stop positions, subsequences of one or both ends of a sequence, and/or lengths) and also have the same molecular barcode.

The present disclosure provides methods and systems for detecting allelic imbalance in a sample from a subject. In an aspect, the present disclosure provides a method for detecting allelic imbalance in a sample from a subject, including: (a) sequencing a plurality of cell-free deoxyribonucleic acid (DNA) molecules from the sample to generate a plurality of sequence reads; (b) aligning at least a portion of the plurality of sequence reads to a reference sequence to produce a plurality of aligned sequence reads; (c) for at least a portion of the plurality of aligned sequence reads, identifying a germline variant present at a mutant allele fraction (MAF) in the sample, thereby identifying a set of germline variants in the sample, wherein individual germline variants in the set of germline variants have corresponding MAF values; (d) determining a quantitative measure of the set of germline variants identified in (c) that are among a plurality of discrete ranges of MAF values; and (e) detecting the allelic imbalance in the sample based on a predetermined criterion by filtering the set of germline variants identified in (c) based on at least the quantitative measure of (d).

In some embodiments, the method further includes: (f) detecting, from the plurality of aligned sequence reads, one or more quantitative measures indicative of copy number variations (CNVs) or diploid genes, wherein the predetermined criterion includes the one or more quantitative measures indicative of the CNVs or the diploid genes.

In some embodiments, the method further includes detecting contamination in the sample when the allelic imbalance is not detected in the sample.

An example method 100 is provided herein. The method 100 may comprise sequencing DNA molecules from a sample for which allelic imbalance or contamination is to be detected, to generate sequence reads (as in operation 102). Next, the method 100 may comprise aligning at least a portion of the sequence reads to a reference sequence, to produce aligned sequence reads (as in operation 104). Next, the method 100 may comprise, for at least a portion of the aligned sequence reads, identifying a set of germline variants in the sample and their corresponding MAF values (as in operation 106), or in certain embodments, identifying corresponding minor allele frequency values. Next, the method 100 may comprise determining a quantitative measure of the germline variants that are among a plurality of discrete ranges of MAF values (as in operation 108), or, in certain embodments, discrete ranges of minor allele frequency values. Next, the method 100 may comprise detecting the allelic imbalance in the sample based on a predetermined criterion by filtering the germline variants based on at least the quantitative measure (as in operation 110). Other examples are found in PCT/US2023/079340, which is fully incorporated by reference herein. Therein, an MR score can be calculated by, for example, comparing driver mutation MAF ratio between one or more time points. For example, for two time points T1 and T1, there is a comparison between maf ratio at T2 and T1 (ave T2 maf/ave T1 maf). Determination therefore include: if MR=1, tumor fraction is not changed; if MR<0.5, tumor fraction significantly decreased; if MR>1, tumor fraction increased. However, if no driver mutations is detected, sample is not evaluable, and it is understood, MR score is affected by CHIP variants. When too many CHIP variants are detected, sample is not evaluable.

The methods and systems provided herein may be particularly useful in the analysis of cell-free nucleic acid molecules (e.g., DNA or RNA molecules). In some cases, cell-free nucleic acid molecules may be extracted and isolated from a readily accessible from a biological sample from a subject. A biological sample may include a bodily fluid sample that is selected from the group including, but not limited to blood, plasma, serum, urine, saliva, mucosal excretions, sputum, stool, and tears. Cell-free nucleic acid molecules can be extracted using a variety of methods, including but not limited to isopropanol precipitation and/or silica-based purification.

The biological sample may be collected from a number of subjects, such as subjects without a disease, subjects at risk for, showing symptoms of, or having a disease, such as cancer or a virus, or subjects at risk for, showing symptoms of, or having a genetic disorder. In some embodiments, the disease or disorder is selected from the group consisting of immune deficiency disorders, hemophilia, thalassemia, sickle cell disease, blood disease, chronic granulomatous disorder, congenital blindness, lysosomal storage disease, muscular dystrophy, cancer, neurodegenerative disease, viral infections, bacterial infections, epidermolysis bullosa, heart disease, fat metabolism disorder, and diabetes, or a combination of these.

After obtaining or providing the cell-free nucleic acids molecules, any of a number of different library preparation procedures for preparing nucleic acid molecules for sequencing may be performed on the cell-free nucleic acid molecules. Cell-free nucleic acid molecules may be processed before sequencing with one or more reagents (e.g., enzymes, adapters, tags (e.g. barcodes), probes, etc.). Tagged molecules may then be used in a downstream application, such as a sequencing reaction by which individual molecules may be tracked.

In some embodiments, the methods may further comprise an enrichment step prior to sequencing, whereby regions of the tagged molecules are selectively or non-selectively enriched.

Once sequencing data of the cell-free nucleic acid molecules is collected, one or more bioinformatics processes may be applied to the sequence data to detect an allelic imbalance or a contamination of the cell-free nucleic acid sample.

In some cases, sequence reads generated from a sequencing reaction can be aligned to a reference sequence for carrying out bioinformatics analysis. In various aspects of bioinformatics analysis, one or more thresholds may be set to ensure quality. For example, an alignment threshold may be set such that only highly similar sequence reads (e.g., with 10 or less mismatches between a reference sequence and sequence reads) are mapped to a reference sequence. In some cases, sequence reads may be removed that cannot pass a quality threshold, e.g. based on chromatograms of sequence reads. In some cases, copy numbers or amounts of a given sequence may be quantified based on the number of sequence reads mapping or aligning to the given sequence. In some cases, over-representation of sequence(s) may be determined by comparing copy numbers or amounts of different sequences among all sequence reads.

In certain embodiments, a sample may be contacted with a sufficient number of adapters that there is a low probability (e.g., less than about 1%, less than about 0.1%, less than about 0.01%, less than about 0.001%, or less than about 0.0001%) that any two copies of the same nucleic acid receive the same combination of adapter molecular barcodes or tags from the adapters linked at one end or both ends. The use of adapters in this manner may permit grouping of sequence reads with the same start and stop points that are aligned (or mapped) to a reference sequence and linked to the same combination of barcodes into families of reads generated from the same original molecule. Such a family may represent sequences of amplification products of a nucleic acid in the sample before amplification.

In some embodiments, sequences of family members can be compiled to derive consensus nucleotide(s) or a complete consensus sequence for a nucleic acid molecule in the original sample, as modified by blunt ending and adapter attachment. In other words, the nucleotide occupying a specified position of a nucleic acid in the sample may be determined to be the consensus of nucleotides occupying that corresponding position in family member sequences. A consensus nucleotide can be determined by methods such as voting or confidence score, to name two non-limiting exemplary methods. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families may include only a single member sequence. In this case, this sequence can be taken as the sequence of a nucleic acid in the sample before amplification. Alternatively, families with only a single member sequence can be eliminated from subsequent analysis.

The reference sequence may be one or more known sequences, e.g., a known whole or partial genome sequence from an object, whole genome sequence of a human object. The reference sequence can be hG19. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeds a threshold, then a variant nucleotide can be called at the designated position. The threshold can be a simple number, such as at least 1, 2, 3, 4, 5, 6, 7, 9, or 10 sequenced nucleic acid within the subset including the nucleotide variant or it can be a ratio, such as a least 0.5, 1, 2, 3, 4, 5, 10, 15, or 20 of sequenced nucleic acids within the subset include the nucleotide variant, among other possibilities. The comparison can be repeated for any designated position of interest in the reference sequence. Sometimes a comparison can be performed for designated positions occupying at least 20, 100, 200, or 300 contiguous positions on a reference sequence, e.g., 20-500, or 50-300 contiguous positions.

The disclosure further provides systems for performing or carrying out the methods described herein. In certain aspects, a system may comprise: (a) a nucleic acid sequencer that generates, as a signal, sequencing reads from adapter-tagged cfDNA molecules from one or more samples, wherein the adapters comprise barcodes that, together with start and stop information from the cfDNA molecule, identify redundant sequence reads from the same original cfDNA molecule; and (b) a computer in communication with the nucleic acid sequencer over a communication network, wherein the computer receives the signal into computer memory and wherein the computer includes a computer processor and computer readable medium including machine-executable code that, upon execution by the computer processor, implements a method including: a) sequencing a plurality of cell-free deoxyribonucleic acid (DNA) molecules from the sample to generate a plurality of sequence reads; b) aligning at least a portion of the plurality of sequence reads to a reference sequence to produce a plurality of aligned sequence reads; c) for each of a plurality of genomic regions, determining, from the plurality of aligned sequence reads, a mutant allele fraction (MAF) of the genomic region in the sample; d) for each of the plurality of genomic regions, determining, from the plurality of aligned sequence reads, whether the genomic region is a germline variant; e) determining a quantitative measure of the determined germline variants of the plurality of genomic regions falling among a plurality of discrete ranges of MAF values; and f) detecting the allelic imbalance in the sample based on a predetermined criterion including the quantitative measure of the determined germline variants.

In some embodiments, the method implemented by the computer processor further includes grouping the sequence reads into families, each of the families including sequence reads including the same barcodes and having the same start and stop positions, whereby each of the families includes sequence reads amplified from the same original cfDNA molecule.

In some embodiments, the sequencer is a DNA sequencer. In some embodiments, the sequencer is designed to perform high-throughput sequencing, such as next generation sequencing. In some embodiments, the system includes adapter tagged cfDNA molecules in the sequencers. In some embodiments, the adapter tagged cfDNA molecules are sourced from one subject or a plurality of subjects. In some embodiments, the cfDNA molecules from the sample bear unique or non-unique barcodes.

### II. General Features of the Methods and Systems

A. Samples

A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equates with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample includes nucleic acids from different sources, e.g., from cells and from cell-free sources (e.g., blood samples, etc.). Typically, a sample include nucleic acids carrying mutations. For example, a sample optionally includes DNA carrying germline mutations and/or somatic mutations. Typically, a sample includes DNA carrying cancer-associated mutations (e.g., cancer-associated somatic mutations).

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides in length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps.
B. Nucleic Acid Tags

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) portions corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcodes, or from about 5 to about 150 different molecular barcodes, or from about 20 to about 50 different molecular barcodes, ligated to both ends of a target molecule. Alternatively, from about 25 to about 1,000,000 different molecular barcodes may be used. For example, 20-50×20-50 molecular barcodes can be used, such that both ends of a target molecules are tagged with one of 20-50 different molecular barcodes. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).
C. Nucleic Acid Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other amplification exemplary methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular barcodes and/or sample indexes to a nucleic acid molecule using conventional nucleic acid amplification methods. The amplifications are typically conducted in one or more reaction mixtures. Molecular barcodes and sample indexes are optionally introduced simultaneously, or in any sequential order. In other embodiments, molecular barcodes and sample indexes are introduced prior to and/or after sequence capturing steps are performed. In some embodiments, only the molecular barcodes are introduced prior to probe capturing and the sample indexes are introduced after sequence capturing steps are performed. In certain embodiments, both the molecular barcodes and the sample indexes are introduced prior to performing probe- based capturing steps. In some embodiments, the sample indexes are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region is associated with a cancer type. Typically, the amplification reactions generate a plurality of non-uniquely or uniquely tagged nucleic acid amplicons with molecular barcodes and sample indexes at a size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.
D. Nucleic Acid Enrichment

In some embodiments, sequences are enriched prior to sequencing the nucleic acids. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic regions associated with the baits, subject to a set of constraints (e.g., sequencer constraints such as sequencing load, utility of each bait, etc.), and capture the targeted nucleic acids at a desired level for downstream sequencing. These targeted genomic regions of interest optionally include natural or synthetic nucleotide sequences of the nucleic acid construct. In some embodiments, biotin-labeled beads with probes to one or more regions of interest can be used to capture target sequences, and optionally followed by amplification of those regions, to enrich for the regions of interest.

Sequence capture typically involves the use of oligonucleotide probes that hybridize to the target nucleic acid sequence. In certain embodiments, a probe set strategy involves tiling the probes across a region of interest. Such probes can be, for example, from about 60 to about 120 nucleotides in length. The set can have a depth of about 2x, 3x, 4x, 5x, 6x, 8x, 9x, 10x, 15x, 20x, 50x or more. The effectiveness of sequence capture generally depends, in part, on the length of the sequence in the target molecule that is complementary (or nearly complementary) to the sequence of the probe.
E. Nucleic Acid Sequencing

Sample nucleic acids, optionally flanked by adapters, with or without prior amplification are generally subject to sequencing. Sequencing methods or commercially available formats that are optionally utilized include, for example, Sanger sequencing, high-throughput sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one or more nucleic acid fragment types or regions known to contain markers of cancer or of other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9% or 100% of the genome. In other cases, sequence reactions may be performed on less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U), which may be present in an easily incorporated form, such as a plurality of nucleoside triphosphates (dNTPs). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including molecular barcodes, and the sequencing determines nucleic acid sequences as well as molecular barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (for e.g., sticky end ligation).

The nucleic acid sample is typically contacted with a sufficient number of adapters that there is a low probability that any two copies of the same nucleic acid receive the same combination of adapter barcodes (i.e., molecular barcodes) from the adapters linked at both ends. The use of adapters in this manner permits identification of families of nucleic acid sequences with the same start and stop points on a reference nucleic acid and linked to the same combination of molecular barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.
F. Analysis

Sequencing according to embodiments of the disclosure generates a plurality of reads. Reads according to the invention generally include sequences of nucleotide data less than about 150 bases in length, or less than about 90 bases in length. In certain embodiments, reads are between about 80 and about 90 bases, e.g., about 85 bases in length. In some embodiments, methods of the invention are applied to very short reads, i.e., less than about 50 or about 30 bases in length. Sequence read data can include the sequence data as well as meta information. Sequence read data can be stored in any suitable file format including, for example, VCF files, FASTA files or FASTQ files.

FASTA is originally a computer program for searching sequence databases and the name FASTA has come to also refer to a standard file format. See Pearson & Lipman, 1988, Improved tools for biological sequence comparison, PNAS 85:2444-2448. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer, as described by, for example, Cock et al. (“The Sanger FASTQ file format for sequences with quality scores, and the Solexa/Illumina FASTQ variants,” Nucleic Acids Res 38(6):1767-1771, 2009), which is hereby incorporated by reference in its entirety.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “—”. In a preferred embodiment, the sequence data will use the A, T, C, G, and N characters, optionally including “—” or U as-needed (e.g., to represent gaps or uracil).

In some embodiments, the at least one master sequence read file and the output file are stored as plain text files (e.g., using encoding such as ASCII; ISO/IEC 646; EBCDIC; UTF-8; or UTF-16). A computer system provided by the invention may include a text editor program capable of opening the plain text files. A text editor program may refer to a computer program capable of presenting contents of a text file (such as a plain text file) on a computer screen, allowing a human to edit the text (e.g., using a monitor, keyboard, and mouse). Exemplary text editors include, without limit, Microsoft Word, emacs, pico, vi, BBEdit, and TextWrangler. Preferably, the text editor program is capable of displaying the plain text files on a computer screen, showing the meta information and the sequence reads in a human-readable format (e.g., not binary encoded but instead using alphanumeric characters as they may be used in print human writing).

While methods have been discussed with reference to FASTA or FASTQ files, methods and systems of the invention may be used to compress any suitable sequence file format including, for example, files in the Variant Call Format (VCF) format. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by Danecek et al. (“The variant call format and VCFtools,” Bioinformatics 27(15):2156-2158, 2011), which is hereby incorporated by reference in its entirety. The header section may be treated as the meta information to write to the compressed files and the data section may be treated as the lines, each of which will be stored in a master file only if unique.

Certain embodiments of the invention provide for the assembly of sequence reads. In assembly by alignment, for example, the reads are aligned to each other or to a reference. By aligning each read, in turn to a reference genome, all of the reads are positioned in relationship to each other to create the assembly. In addition, aligning or mapping the sequence read to a reference sequence can also be used to identify variant sequences within the sequence read. Identifying variant sequences can be used in combination with the methods and systems described herein to further aid in the diagnosis or prognosis of a disease or condition, or for guiding treatment decisions.

In some embodiments, any or all of the steps are automated. Alternatively, methods of the invention may be embodied wholly or partially in one or more dedicated programs, for example, each optionally written in a compiled language such as C++ then compiled and distributed as a binary. Methods of the invention may be implemented wholly or in part as modules within, or by invoking functionality within, existing sequence analysis platforms. In certain embodiments, methods of the invention include a number of steps that are all invoked automatically responsive to a single starting cue (e.g., one or a combination of triggering events sourced from human activity, another computer program, or a machine). Thus, the invention provides methods in which any or the steps or any combination of the steps can occur automatically responsive to a cue. Automatically generally means without intervening human input, influence, or interaction (i.e., responsive only to original or pre-cue human activity).

The system also encompasses various forms of output, which includes an accurate and sensitive interpretation of the subject nucleic acid. The output of retrieval can be provided in the format of a computer file. In certain embodiments, the output is a FASTA file, FASTQ file, or VCF file. Output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact Idiosyncratic Gapped Alignment Report (CIGAR) (Ning et al., Genome Research 11(10):1725-9, 2001, which is hereby incorporated by reference in its entirety). These strings are implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file—including a CIGAR string (the SAM format is described, e.g., by Li et al., “The Sequence Alignment/Map format and SAMtools,” Bioinformatics, 25(16):2078-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save some space), 3 matches, 2 deletions and 2 matches.

In some embodiments, the results of the systems and methods disclosed herein are used as an input to generate a report. The report may be in a paper or electronic format. For example, information on the allelic imbalance status of a sample determined by the methods or systems disclosed herein can be displayed in such a report. Alternatively or additionally, information on the presence or absence of contamination in the sample, as determined by the methods or systems disclosed herein, can be displayed in such a report. The methods or systems disclosed herein may further comprise a step of communicating the report to a third party, such as the subject from whom the sample derived or a health care practitioner.

The various steps of the methods disclosed herein, or the steps carried out by the systems disclosed herein, may be carried out at the same or different times, in the same or different geographical locations, e.g., countries, and/or by the same or different people.

The present methods can be also used for determining or monitoring the efficacy of the treatment by the relative amounts of the therapeutic nucleic acid construct at different time points.

In various embodiments, this includes a computer system that is programmed or otherwise configured to implement methods provided herein.

The computer system may be programmed or otherwise configured to implement architectures for training neural networks using biological sequences, conservation, and molecular phenotypes. The computer system can regulate various aspects of the present disclosure, such as, for example, (a) sequencing a plurality of cell-free deoxyribonucleic acid (DNA) molecules from the sample to generate a plurality of sequence reads; (b) aligning at least a portion of the plurality of sequence reads to a reference sequence to produce a plurality of aligned sequence reads; (c) for at least a portion of the plurality of aligned sequence reads, identifying a germline variant present at a mutant allele fraction (MAF) in the sample, thereby identifying a set of germline variants in the sample, wherein individual germline variants in the set of germline variants have corresponding MAF values; (d) determining a quantitative measure of the set of germline variants identified in (c) that are among a plurality of discrete ranges of MAF values; and (e) detecting the allelic imbalance in the sample based on a predetermined criterion by filtering the set of germline variants identified in (c) based on at least the quantitative measure of (d). The computer system 301 can be an electronic device of a user or a computer system that is remotely located with respect to the electronic device. The electronic device can be a mobile electronic device.

The computer system includes a central processing unit (CPU, also “processor” and “computer processor” herein), which can be a single core or multi core processor, or a plurality of processors for parallel processing. The computer system 301 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 301 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that includes a user interface (UI). Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, (a) align at least a portion of a plurality of sequence reads from a sequencer to a reference sequence to produce a plurality of aligned sequence reads; (b) for at least a portion of the plurality of aligned sequence reads, identify a germline variant present at a mutant allele fraction (MAF) or minor allele frequency in a sample, thereby identifying a set of germline variants in the sample, wherein individual germline variants in the set of germline variants have corresponding MAF or minor allele frequency values; (c) determine a quantitative measure of the set of germline variants identified in (b) that are among a plurality of discrete ranges of MAF or minor allele frequency values; and (d) detect the allelic imbalance in the sample based on a predetermined criterion by filtering the set of germline variants identified in (b) based on at least the quantitative measure of (c).

Although the description has been described with respect to particular embodiments thereof, these particular embodiments are merely illustrative, and not restrictive. Concepts illustrated in the examples may be applied to other examples and implementations.

### EXAMPLES

### Example 1: AI MR Score

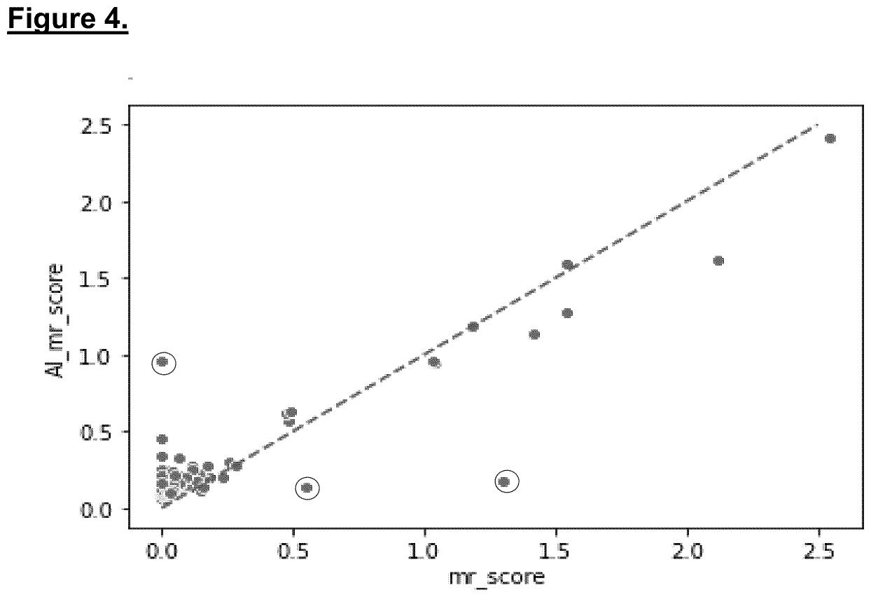

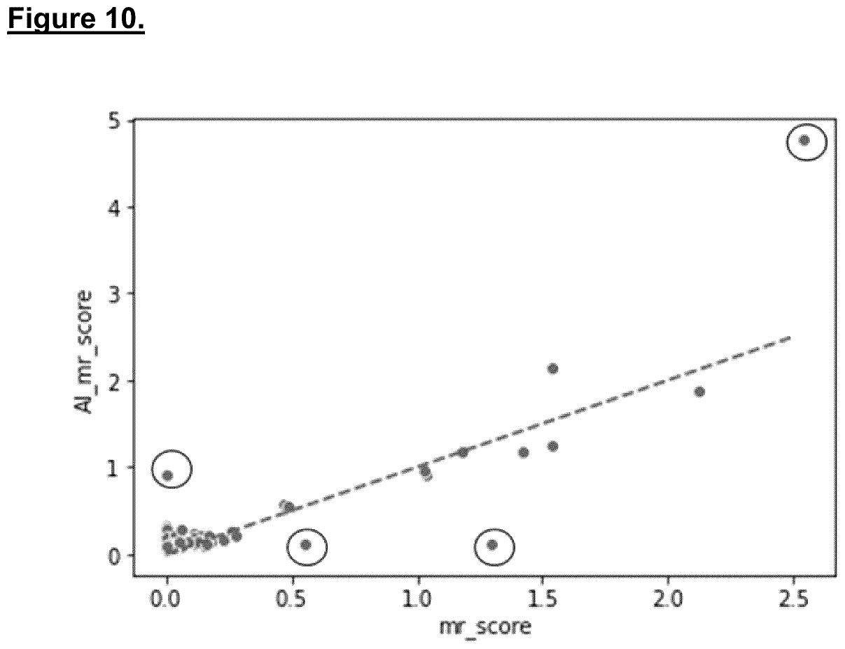

Briefly, an exemplary approach includes selecting common SNPs with maf in [5%, 95%] (indels are excluded), followed by select allelic imbalance SNPs (maf<45% or maf>55% & cluster of allelic imbalance (n>=5)). Thereafter, one can calculate the maf shift score for each SNPs: abs(maf-50%). This is followed by gene-level maf shift score: the median maf shift score of all the SNPs in the same gene. Together, this allows determination of an AI MR score, wherein AI MR=median(gene-level maf shift score at T1)/median(gene-level maf shift score at T0). This has been validated in results demonstrating that AI MR score and MR score are similar in most samples, as shown in FIGS. 4 and 10.

### Example 2: Example Shift Score

More specifically, an MAF shift score is calculated for each SNP from the set of SNPs including MAFs[5%-95%]. The MAF shift score for each SNPs is the absolute value of the difference between the SNP's MAF and 50%, i.e., the magnitude of the deviation from 50%.

Thereafter, a gene-level MAF shift score, which is the median MAF shift score of all the SNPs in the same gene, is determined. The Ai MR score is the relative change in the median gene-level MAF shift score from T=0 to t=1 with examples being Ai MR score=1 tumor fraction is not changed; Ai MR score<0.5, tumor fraction significantly decreased; Ai MR score>1, tumor fraction significantly increased.

### Example 3: Increase Evaluable Samples

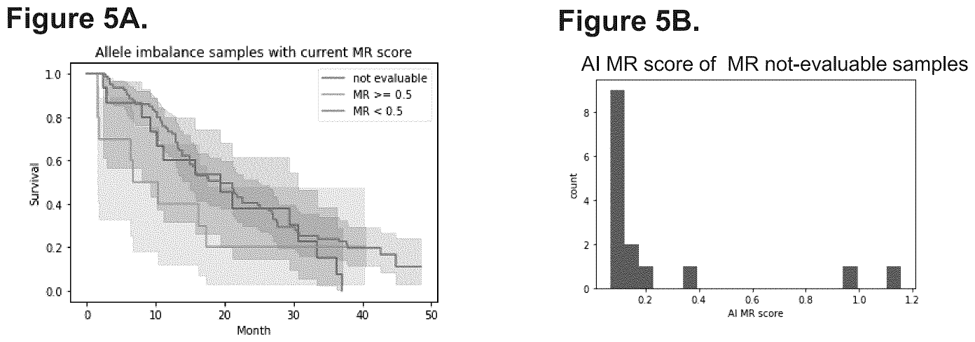

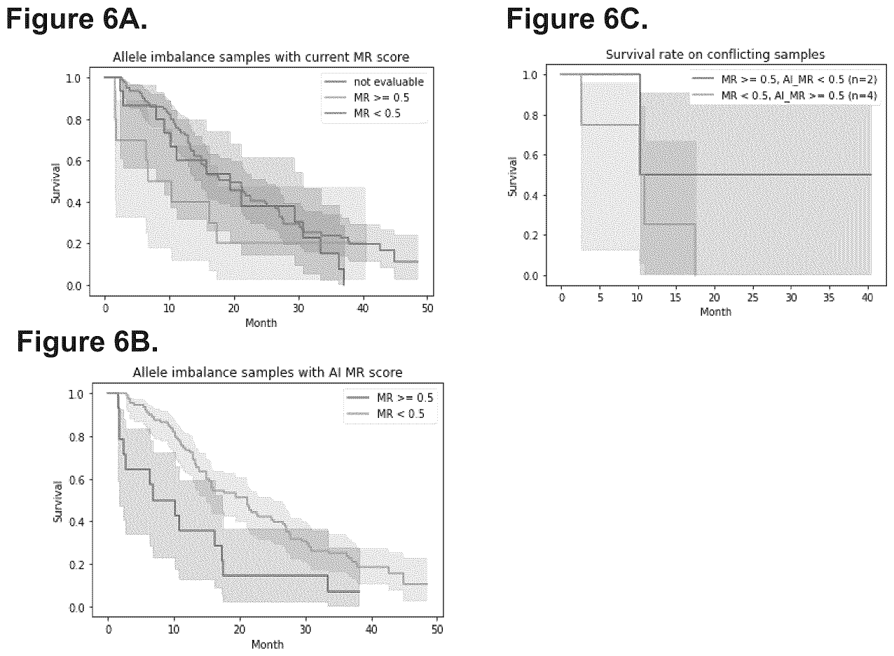

As an exemplary illustration of improving evaluable samples, a breast cancer cohort included 373 samples, with 135 samples are not evaluable, 104 samples with allelic imbalance and 15 not evaluable samples can be evaluabled by AI MR Some MR not-evaluable samples have high AI MR score, FIGS. 5 and 6.

### Example 4: AI MR CNV Score

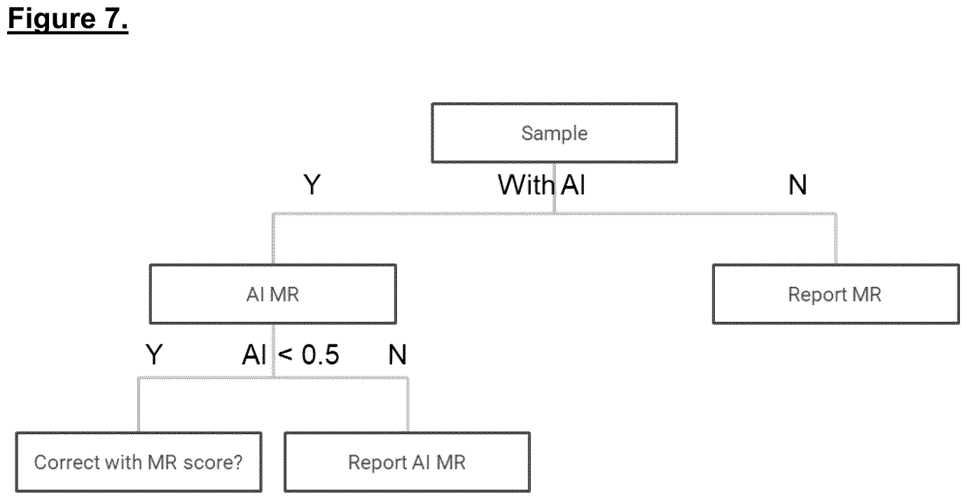

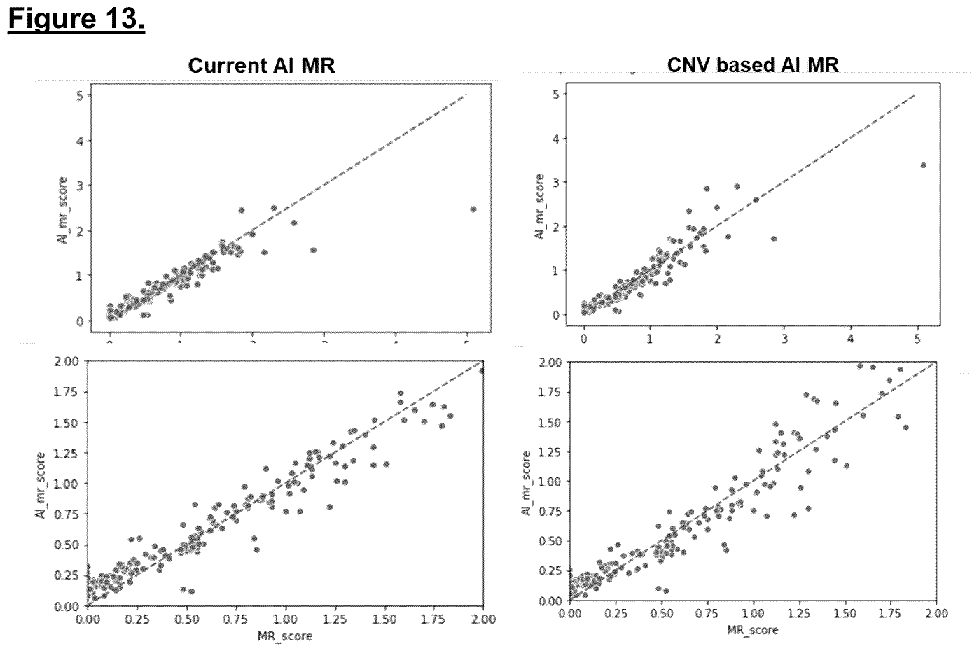

Alternatively, one can calculate tumor fraction changes based on maf1 and maf0. The new method, shown in Formula 1, the AI MR-CNV score is the median tumor fraction changes in gene level. In most cases, AI MR-CNV has the same results as AI MR as shown in FIG. 13. In some instances, a composite final score will be based on all the methods (MR, AI MR, AI MR-CNV), an example is shown in FIG. 7 . . .

This is based on the usual observation that a SNP in 1 allele, has a maf of 50%, and when there is a duplication in the allele without the SNP and the copy number is 2, the maf in the tumor cell will become 1 (copies)/3 (total copies). If considers tumor fraction. the cancer cell have tumor fraction=f, with mutant allele=f*1, total allele=f*n, the normal cell have (1−f) in the total cfDNA, with mutant allele=(1−f)*1 and total allele=(1−f)*2. Therefore, the observed maf=(f*1+(1−f)*1)/(f*n+(1−f)*2).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer implemented method comprising:
receiving a plurality of sequence reads from a subject;
aligning the sequence reads to a reference;
determining one or more metrics based on the alignment to the reference; and
processing the one or more metrics generated from the aligned reads to determine a sample score.

2. The method of claim 1, further comprising:
obtaining a sample from the subject; and
sequencing the sample to obtain the plurality of sequence reads.

3. The method of claim 1, wherein the one or more metrics comprise a plurality of single nucleotide polymorphisms (SNPs).

4. The method of claim 3, wherein the SNPs comprise common SNPs.

5. The method of claim 3, wherein the SNPs have a minor allele frequency (MAF) ranging from 5% to 95%.

6. The method of claim 3, further comprising determining, from the plurality of SNPs, at least one allelic imbalance cluster.

7. The method of claim 6, wherein the at least one allelic imbalance cluster comprise SNPs with MAF less than 45% or greater than 55%.

8. The method of claim 6, wherein the at least one allelic imbalance cluster comprise SNPs with a MAF either less than 50% or greater than 50%.

9. The method of any one of claims 6 to 8, wherein the at least one allelic imbalance clusters comprise at least one SNPs.

10. The method of any one of claims 6 to 8, wherein the at least one allelic imbalance clusters comprise five or more SNPs.

11. The method of any one of the preceding claims, wherein the sample score comprises determining a MAF shift score for each SNP.

12. The method of claim 11, wherein the MAF shift score comprises the absolute difference between MAF for a given SNP and 50%.

13. The method of any one of claim 11 or 12, further comprising: determining, a gene-level MAF shift score.

14. The method of claim 13, wherein the gene-level MAF shift score comprises the median, second quartile, mean, weighted mean, geometric mean, harmonic mean, or winsorized mean MAF shift score of all the SNPs in the same gene.

15. The method of claim 1, wherein the sequence reads comprise data from a plurality of samples collected at different timepoints from the same subject.

16. The method of claim 1 comprising: determining an allelic imbalance molecular response score (Ai MR score).

17. (canceled)

18. (canceled)

19. (canceled)

20. (canceled)

21. (canceled)

22. (canceled)

23. The method of claim 1, wherein the sample comprises cell free nucleic acids (cf NA).

24. The method of claim 23, wherein the cf NA are cell free DNA or cell free RNA.

25. (canceled)

26. (canceled)

27. A computer implemented method comprising:
receiving a plurality of sequence reads comprising data from a plurality of samples from the same subject;
aligning the sequence reads to a reference;
processing the sequence reads to determine a plurality of allelic imbalance clusters comprising at least one SNP;
determining a MAF shift score for each SNP in the cluster;
determining a gene-level MAF shift scores for all the SNPs in the same gene; and processing the gene-level MAF shift scores to determine an allelic imbalance molecular response score (Ai MR score) for the plurality of samples from the patient.

28. The method of claim 27, further comprising:
obtaining a plurality of samples from the subject, wherein at least one sample from the plurality of samples is obtained before administering a therapy to the subject; and
sequencing the plurality of samples to obtain the plurality of sequence reads.

29. The method of claim 28, wherein the therapy is selected from the group consisting of an immune checkpoint inhibitor, poly (ADP-ribose) polymerase (PARP) inhibitor, a kinase inhibitor, or an aromatase inhibitor, a PI3K and mTOR inhibitor.

30. The method of claim 29, wherein the immune checkpoint inhibitor comprises Pembrolizumab.

31. The method of claim 29, wherein the poly (ADP-ribose) polymerase (PARP) inhibitor Olaparib or Talazoparib.

32. The method of claim 29, wherein the therapy is a combination of a PI3K and mTOR inhibitor and a poly (ADP-ribose) polymerase (PARP) inhibitor.

33. The method of claim 32, wherein the PI3K and mTOR inhibitor comprises Gedatolisib and the poly (ADP-ribose) polymerase (PARP) inhibitor comprises Talazoparib.

34. The method of claim 27, further comprising determining a therapeutic response in the subject when the Ai MR score falls below a predefined threshold.

35. The method of claim 29, wherein Ai MR score of 1 indicates unchanged tumor fraction, Ai MR score greater than 1 indicates increased tumor fraction, and Ai MR less than 1 indicates decrease tumor fraction.

36. The method of claim 29, wherein an Ai MR less than 0.5 indicates a therapeutic response.

37. The method of claim 29, wherein an Ai MR less than 1 indicates a therapeutic response.

38. (canceled)
